(19) United States
(12) Patent Application Publication
(10) Pub. No.: US 2014/0316218 A1
(43) Pub. Date: Oct. 23, 2014

(54) SYSTEMS AND METHODS FOR MONITORING BRAIN METABOLISM AND ACTIVITY USING ELECTROENCEPHALOGRAM AND OPTICAL IMAGING

(71) Applicants: Patrick L. Purdon, Somerville, MA (US); Emery N. Brown, Brookline, MA (US); ShiNung Ching, Cambridge, MA (US); David A. Boas, Winchester, MA (US); Maria Angela Franceschini, Winchester, MA (US); Jason Sutin, Cambridge, MA (US)

(72) Inventors: Patrick L. Purdon, Somerville, MA (US); Emery N. Brown, Brookline, MA (US); ShiNung Ching, Cambridge, MA (US); David A. Boas, Winchester, MA (US); Maria Angela Franceschini, Winchester, MA (US); Jason Sutin, Cambridge, MA (US)

(21) Appl. No.: 14/260,070

(22) Filed: Apr. 23, 2014

Related U.S. Application Data
(60) Provisional application No. 61/815,144, filed on Apr. 23, 2013.

(51) Int. Cl. A61B 5/00; A61B 5/1455; A61B 5/026

(52) U.S. Cl. CPC A61B 5/4866; A61B 5/0075; A61B 5/0261; A61B 5/4064; A61B 5/14553
USPC 600/301; 600/544; 607/96

(57) ABSTRACT

Systems and methods for monitoring and/or controlling a brain state of a subject are provided. In certain embodiments, the method includes acquiring physiological data from sensors including electrophysiological sensors and optical sensors, assembling, using data from the electrophysiological sensors, a time-series signal indicative of a brain activity of the subject, and identifying, using the time-series signal, a burst suppression state described by a burst suppression period and a burst period. The method also includes computing, using data from the optical sensors, parameters associated with the burst suppression state, the parameters indicative of least one of a metabolic process and a hemodynamic process, and estimating, using the parameters, time-series signal, and burst period, a response function describing a time course of the parameters correlated with a burst during the burst suppression period. The method further includes controlling a treatment using the response function to generate a target burst suppression state.

### CROSS-REFERENCE TO RELATED APPLICATIONS

This application is based on, claims priority to, and incorporates herein by reference in its entirety, U.S. Provisional Application Ser. No. 61/815,144, filed Apr. 23, 2013, and entitled “A System and Method for Monitoring Brain Metabolism and Activity in the Operating Room and Intensive Care Unit Using Electroencephalogram and Near Infrared Spectroscopy.”

### STATEMENT REGARDING FEDERALLY SPONSORED RESEARCH

This invention was made with government support under grants TR01-GM104948, DP2-OD006454, P41-RR14075, R01-EB001954, R01-EB002482, R01-EB006385 awarded by the National Institutes of Health. The government has certain rights in the invention.

### BACKGROUND OF THE INVENTION

The present disclosure generally relates to systems and method for monitoring and controlling a state of a subject and, more particularly, to systems and methods directed monitoring and controlling a subject using measures of brain metabolism and activity.

More that 75 years ago it was demonstrated that central nervous system changes, as occurring when subjects received increasing doses of either ether or pentobarbital, were observable via electroencephalogram (“EEG”) recordings, which measure electrical impulses in the brain through electrodes placed on the scalp. As a consequence, it was postulated that the electroencephalogram could be used as a tool to track in real time the brain states of subjects under sedation and general anesthesia, the same way that an electrocardiogram (“ECG”) could be used to track the state of the heart and the cardiovascular system.

Common brain activity that clinicians encounter include periods of “burst suppression,” which is an example of an EEG pattern that can be observed when the brain has severely reduced levels of neuronal activity, metabolic rate, and oxygen consumption. The burst suppression pattern often manifests as periods of bursts of electrical activity alternating with periods during which the EEG is isoelectric or suppressed, and may typically be the result of injuries, disorders, or medical interventions. For example, burst suppression is commonly seen in profound states of general anesthesia, such as a medically-induced coma.

A variety of clinical scenarios require controlling the brain state of a subject for purposes of brain protection, including treatment of uncontrolled seizures—status epilepticus—and brain protection following traumatic or hypoxic brain injury, anoxic brain injuries, hypothermia, and certain developmental disorders. For example, since burst suppression represents a specific brain state when the brain is in an altered metabolic state, it is commonly targeted using anesthetic drugs, such as propofol, in order to protect the brain. Similarly, during major cardiac surgery, subjects are sometimes placed into deep burst suppression through hypothermia, also offering brain protection through reduced metabolism. In addition, cardiac arrest subjects are similarly placed into burst suppression via hypothermia for brain protection.

Therefore, indicators of brain metabolism are desirable to help ensure adequate levels of burst suppression, such as during administration of an anesthetic compound or a hypothermia treatment. Such brain metabolism information may offer prognostic indications on a likelihood or trajectory of recovery, or on the efficacy of drug therapies and other interventions designed to speed or enhance recovery.

Similarly, cerebral hemodynamic response to metabolism could also be altered under different pathological or medical circumstances. Hence, hemodynamic responses during burst suppression, as well as flow-metabolism coupling ratios, and their variations with time, temperature, drug concentration, and other interventions, are also desirable for diagnostic purposes in these settings.

Hence, considering the above, there continues to be a clear need for systems and methods to accurately monitor subject states and based thereon, provide systems and methods for controlling subject states.

### SUMMARY OF THE INVENTION

The present disclosure overcomes shortcomings of previous technologies by providing systems and methods directed to monitoring and controlling a state of a subject. Specifically, a novel approach is introduced that makes use of electroencephalogram (“EEG”) and optical imaging measures to precisely determine indications with respect to brain metabolism and activity during specific brain states of a subject, such as a neurophysiological state of burst suppression, for purposes of monitoring and controlling a brain state of a subject. Systems and methods, as will be described, may be applied specifically in settings associated with general anesthesia, deep sedation during intensive care, medically-induced coma, hypothermia, brain injury, or other pathology

In one aspect of the present disclosure, a system for monitoring and controlling a state of a subject is provided. The system includes an input configured to receive physiological data from a plurality of sensors coupled to the subject, the plurality of sensors including electrophysiological sensors and optical sensors, and at least one optical source configured to direct light in a range of wavelengths to at least one portion of a subject's anatomy. The system also includes at least one processor configured acquire the physiological data from the plurality of sensors positioned on the subject, assemble, using the physiological data from the electrophysiological sensors, a time-series signal indicative of a brain activity of the subject, and identify, using the time-series signal, a burst suppression state described by a burst suppression period and a burst period. The at least one processor is also configured to compute, using the physiological data from the optical sensors, parameters associated with the burst suppression state, the parameters indicative of least one of a metabolic process and a hemodynamic process, and estimate, using the parameters, time-series signal, and burst period, a response function describing a time course of the parameters correlated with a burst during the burst suppression period. The at least one processor is further configured to generate a report indicative of the response function.

In another aspect of the present disclosure, a method for monitoring a brain state of a subject is provided. The method includes acquiring physiological data from a plurality of sensors positioned on the subject, the plurality of sensors including electrophysiological sensors and optical sensors, assembling, using the physiological data from the electrophysiological sensors, a time-series signal indicative of a brain activity of the subject, and identifying, using the time-series signal, a burst suppression state described by a burst suppression period and a burst period. The method also includes computing, using the physiological data from the optical sensors, parameters associated with the burst suppression state, the parameters indicative of least one of a metabolic process and a hemodynamic process, and estimating, using the parameters, time-series signal, and burst period, a response function describing a time course of the parameters correlated with a burst during the burst suppression period. The method further includes generating a report indicative of the response function.

In yet another aspect of the present disclosure, a method for monitoring and controlling a brain state of a subject. The method includes acquiring physiological data from a plurality of sensors positioned on the subject, the plurality of sensors including electrophysiological sensors and optical sensors, assembling, using the physiological data from the electrophysiological sensors, a time-series signal indicative of a brain activity of the subject, and identifying, using the time-series signal, a burst suppression state described by a burst suppression period and a burst period. The method also includes computing, using the physiological data from the optical sensors, parameters associated with the burst suppression state, the parameters indicative of least one of a metabolic process and a hemodynamic process, and estimating, using the parameters, time-series signal, and burst period, a response function describing a time course of the parameters correlated with a burst during the burst suppression period. The method further includes controlling an administration of a treatment using the response function to achieve a target burst suppression state.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will hereafter be described with reference to the accompanying drawings, wherein like reference numerals denote like elements.

FIGS. 1A and 1B are schematic block diagrams of example physiological monitoring systems.

FIG. 2 is a schematic diagram illustrating a process of combining physiological data acquired using optical imaging methods.

FIG. 3 is a block diagram of an example system for use in accordance with the present disclosure.

FIG. 4A is an illustration of an example monitoring and control system in accordance with the present disclosure.

FIG. 4B is an illustration of an example sensor array for the system of FIG. 4A.

FIG. 5 is a flow chart setting forth the steps of a monitoring and control method in accordance with the present disclosure.

FIG. 6 is an illustration of an example sensor configuration.

FIG. 7 shows a time-series example of metabolic parameters in relation to time-series EEG data during the transition from 1 to 2% isoflurane.

FIG. 8A shows another time-series example of metabolic and hemodynamic parameters in relation to time-series EEG data.

FIG. 8B shows a time-series example of measured relative cerebral blood flow in comparison to fitted responses obtained using a method in accordance with the present disclosure.

FIG. 9 shows a graphical illustration of estimated burst-suppression HRF's for HbO, HbR, HbT, CBF, and CMRO₂.

FIG. 10 shows a graphical illustration of measured metabolic parameters and respective estimated HRF during increasing (top) and decreasing (bottom) concentrations of isoflurane.

FIG. 11 shows a graphical illustration of measured hemoglobin parameters and oxygen metabolism functional relative changes parameter during increasing concentrations of isoflurane.

### DETAILED DESCRIPTION

Burst suppression is an electroencepholagram (“EEG”) pattern in which high-voltage activity alternates with isoelectric quiescence. It is characteristic of an inactivated brain and is commonly observed at deep levels of general anesthesia, hypothermia, and in pathological conditions such as coma and early infantile encephalopathy. Recently, a unifying mechanism has been proposed for burst suppression, whereby, using a biophysical computational model, prevailing features of burst suppression were shown to arise through the interaction between neuronal dynamics and brain metabolism. Specifically, these features include a synchrony of bursts onset across a subject's scalp, a parametric sensitivity to the level of brain depression, such as a depth of anesthesia, and timescales associated with burst suppressions occurring much slower than other neural activity. In each condition, the model suggested that a decrease in cerebral metabolic rate, coupled with the stabilizing properties of ATP-gated potassium channels, could lead to the characteristic epochs of suppression underlying the burst suppression EEG pattern.

As will be described, the present invention recognizes that indicators related to metabolic and hemodynamic processes may be combined with measures of electrical brain activity, among others, to monitor and control a brain state of a subject. Specifically, EEG data and optical imaging data, acquired substantially simultaneously, may be used to monitor brain metabolism and hemodynamic responses associated with a neurophysiological state of burst suppression. In particular, using parameters determined from acquired optical data, response functions describing a time course for parameters as correlated with a burst during the burst suppression period may be determined. For example, these parameters may include hemoglobin baseline values, hemoglobin functional changes, hemoglobin functional relative changes, blood flow functional relative changes, oxygen metabolism functional relative changes, and so on. Therefore, such measurements may be used to quantify a current brain state, as well as offer prognostic information for determining and/or controlling a future brain state of the subject. For example, such information may be used to provide indications of a trajectory of a recovery, or an efficacy of treatment, or a depth of anesthesia or sedation.

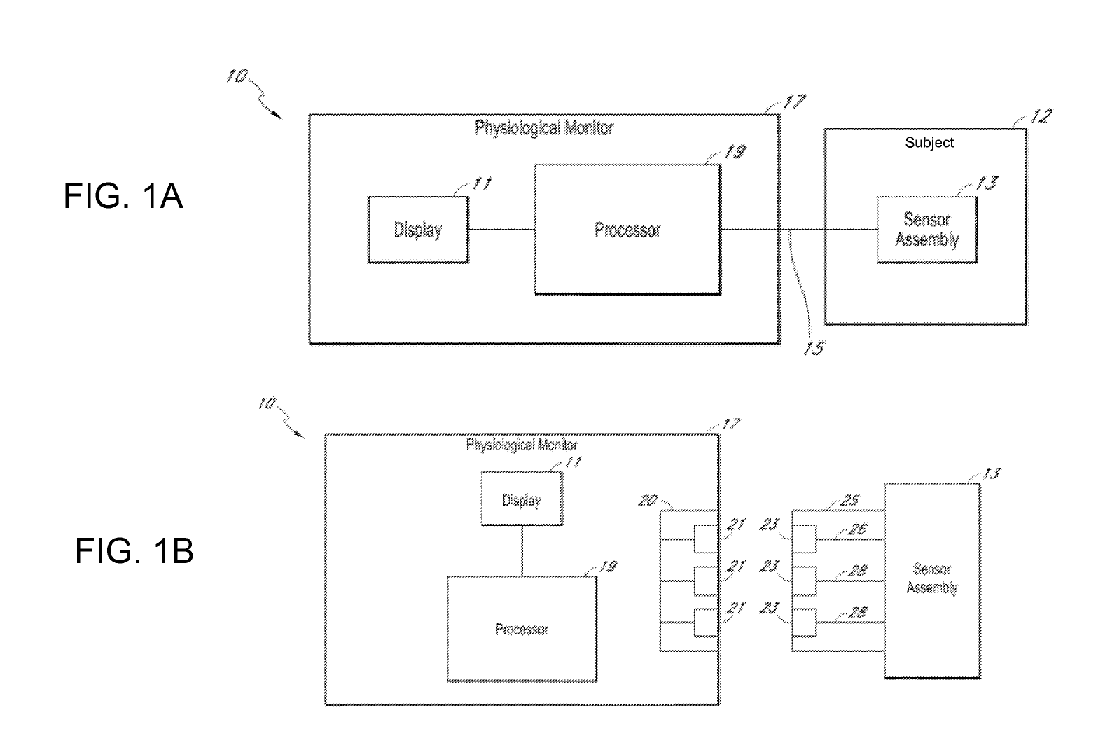

Referring specifically to the drawings, FIGS. 1A and 1B illustrate example subject monitoring systems and sensors that can be used to provide physiological measures of a subject, for use in providing indications of a brain state of a subject.

For example, FIG. 1A shows an embodiment of a physiological monitoring system 10. In the physiological monitoring system 10, a subject 12 is monitored using a sensor assembly 13 that includes one or more sensors, each of which transmits a signal over a cable 15 or other communication link or medium to a physiological monitor 17. The physiological monitor 17 includes a processor 19 and, optionally, a display 11.

Specifically, the sensor assembly 13 includes sensing elements, such as, for example, electrophysiological sensors, such as EEG sensors, optical sensors, such as blood oxygenation sensors, ECG sensors, temperature sensors, acoustic respiration monitoring sensors, and so forth. The sensor assembly 13 may also include features and elements configured as appropriate for positioning, or fastening the sensor assembly 13 to any desired portion of a subject's anatomy, such a scalp or forehead. Each of the sensors can generate respective signals by measuring any number of physiological parameters associated with the subject 12, as well as other parameters. In some configurations, the sensor array 13 may include at least EEG sensors and optical sensors, with additional sensors of different types optionally included. Other combinations of numbers and types of sensors are also suitable for use with the physiological monitoring system 10.

The sensor assembly 13 may be configured with one or more optical sources designed to generate light in a range of wavelengths and direct the generated light to any desirable portion of a subject's anatomy. For example, the range of wavelengths may include a near-infrared range between 650 and 950 nanometers, although other values are possible. The optical source(s) may include one or more emitters or emitter systems, and such emitters or emitter systems may be embedded into a substrate. In various configurations, the emitters could be either light emitting diodes (“LEDs”), lasers, superluminescent LEDs or some other light emitting components. These components could be arranged in any pattern on the substrate and could be either a single light emitting source or several light emitting sources.

The signals generated by the sensors 13 are then processed by one or more processors 19. The one or more processors 19 then communicate the processed signal to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

In some embodiments of the system shown in FIG. 1A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensor assembly 13.

As shown in FIG. 1B, the sensor assembly 13 can include a cable 25. The cable 25 can include three conductors within an electrical shielding. One conductor 26 can provide power to a physiological monitor 17, one conductor 28 can provide a ground signal to the physiological monitor 17, and one conductor 28 can transmit signals from the sensor assembly 13 to the physiological monitor 17. For multiple sensors, one or more additional cables 15 can be provided.

In some embodiments, the ground signal is an earth ground, but in other embodiments, the ground signal is a subject ground, sometimes referred to as a subject reference, a subject reference signal, a return, or a subject return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor assembly 13 and the physiological monitor 17 communicate wirelessly.

In addition to other processing steps for operating the physiological monitoring system 10, the one or more processors 19 may be configured to determine hemodynamic and metabolic parameters, as will be described, by processing physiological data acquired from optical sensors. In some aspects, determined parameters are associated with a particular brain state of a subject, such as a burst suppression state. Furthermore, as will be described, the one or more processors 19 may also be configured to estimate, using the computed parameters, response functions describing a time course of the parameters correlated with a burst during the burst suppression period.

In some embodiments of the present disclosure, the monitoring system 10 may be configured to acquire physiological data from a subject and determine hemoglobin baseline values therefrom using a frequency domain near infra-red spectroscopy (“FD-NIRS”) technique. Specifically, analysis of FD-NIRS data may include data quality assessment and data rejection based on pre-determined statistical criteria. As such, amplitude and phase information may be collected from FD-NIRS data acquired at any number different wavelengths, by way of optical sensors, or detectors, placed at any number of distances away from the optical sources to determine absorption and scattering coefficients. In this manner, baseline oxygenated and deoxygenated hemoglobin concentrations (HbO and HbR, respectively) may be determined by fitting the absorption coefficients at all wavelengths with the hemoglobin spectra. Total hemoglobin (HbT=HbO+HbR), hemoglobin oxygenation (SO₂=HbO/HbT), and cerebral blood volume may also be derived using the hemoglobin baseline values.

In some embodiments of the present disclosure, the monitoring system 10 may be configured to acquire physiological data from a subject and determine hemoglobin functional changes therefrom using a continuous-wave near-infrared spectroscopy (“CW-NIRS”) technique. In some aspects, CWNIRS data may be pre-processed, for example, using band-pass filtering with filters in a frequency range between 0.016 and 0.6 Hz, although other values are possible. In addition, PCA-based filtering (for example, using, say, an 80% threshold value) may be applied to reduce motion artifacts. Residual movement artifacts may rejected using an automated detection algorithm based on standard deviation. Specifically, block averages over any period from stimuli onsets, say in a range between −5 and +25 second although other values may be possible, may be performed and changes in optical density for any source-detector pair may then be converted to changes in hemoglobin concentration (ΔHbO, ΔHbR, ΔHbT) using a modified Beer-Lambert relationship. The differential pathlength factor (“DPF”) can be calculated using the FDNIRS-measured absorption coefficients and average scattering coefficients, as described. Relative changes in blood oxygenation and volume may then be derived by combining hemoglobin FDNIRS baseline values and CWNIRS functional changes:

$$\begin{matrix}{SO}_{2} ( t ) = \frac{{HbO}_{FDNIRS} + Δ {HbO}_{CWNIRS} ( t )}{{HbT}_{FDNIRS} + Δ {HbT}_{CWNIRS} ( t )} & ( 1 ) \\ {rSO}_{2} = \frac{{SO}_{2} ( t )}{{SO}_{2} ( {t}_{0} )} & ( 2 ) \\ HbT ( t ) = {HbT}_{FDNIRS} + Δ {HbT}_{CWNIRS} ( t ) & ( 3 ) \\ rCBV = \frac{HbT ( t )}{Hbt ( {t}_{0} )} & ( 4 )\end{matrix}$$

In some embodiments of the present disclosure, the monitoring system 10 may be configured to acquire physiological data from a subject and determine blood flow functional relative changes therefrom using a diffusion correlation spectroscopy (“DCS”) technique. Diffuse correlation spectroscopy offers a measure of tissue perfusion that depends on both the movement of scatterers inside the blood vessels and the tissue optical properties. For example, tissue optical properties may be derived from the FDNIRS baseline data, acquired say using 785 nm wavelength light, although other values are possible. Regarding determination of the DPF. FDNIRS-measured absorption coefficients and average scattering coefficients, as described, may be used.

DCS intensity auto-correlation curves (for example, over a delay time range of 200 ns˜1 s) acquired substantially sequentially, say, once per second, may be fitted to the normalized intensity temporal auto-correlation function to obtain a blood flow index (BFᵢ). To estimate relative changes in blood flow an analysis similar to that used for the CWNIRS data may be used. For example, a 0.016-Hz high-pass filter may be applied to the BFᵢ normalized data, along with removal of movement artifacts, and block averaged the data over a time period around the stimuli in a range between −5 to +25 sec. The relative changes in cerebral blood flow may then be calculated as:

$$rCBF = \frac{B {F}_{i} ( t )}{B {F}_{i} ( {t}_{0} )} \qquad ( 5 )$$

By combining relative changes in blood flow and oxygenation obtained from FDNIRS, CWNIRS and DCS data, the relative cerebral metabolic rate of oxygen may be estimated as follows:

rCMRO₂=rCBF×rOEF (6)

where OEF is the oxygen extraction fraction:

$$r O E F = \frac{{Sa O}_{2} ( t ) - {Sv O}_{2} ( t )}{{Sa O}_{2} ( {t}_{0} ) - {Sv O}_{2} ( {t}_{0} )} = \frac{{Sa O}_{2} ( t ) - {S O}_{2} ( t )}{{Sa O}_{2} ( {t}_{0} ) - {S O}_{2} ( {t}_{0} )} \qquad ( 7 )$$

with venous oxygenation

SvO₂=(SO₂−a×SaO₂)/b (8)

with a+b=1, a and b the arterial and venous contributions constant over time, and arterial oxygenation SaO₂=100%.

In addition to the steady-state formulation above, the rCMRO₂ may be calculated using two additional models. The first model, allows assignment of different fractions of functional changes versus baseline values of HbR and HbT concentrations in the venous compartment with respect to the total volume fractions:

$$rC M R {O}_{2} = ( 1 + {γ}_{r} \frac{Δ HbR}{HbR} ) \times {( 1 + {γ}_{t} \frac{Δ HbT}{HbT} )}^{- 1} \times ( \frac{Δ CBF + CBF}{CBF} ) \qquad ( 9 )$$

where γᵣ and γₜ are constants used to assign different weights to the venous compartment:

$${γ}_{r} = \frac{Δ {HbR}_{v} / Δ HbR}{{HbR}_{v} / HbR} and$$

$${γ}_{t} = \frac{Δ {HbT}_{v} / Δ HbT}{{HbT}_{v} / HbT}$$

where γᵣ and γₜ may be in a range between 0.5 to 2, and more specifically in a range between 0.75 to 1.25. Under the assumption SaO₂=100%, for γᵣ and γₜ equal to 1, Eqn. (8) reduces to Eqn. (6).

The second model, allows testing for the influence of the blood transit time from the arterial to venous compartment on the oxygen extraction fraction. Using this model rOEF may be calculated as follows:

$$rOEF = \frac{rHbR}{rCBV} + \frac{τ}{rCBF} ( Δ rHbR - \frac{rHbR}{rCBV} \times Δ rCBV ) \qquad ( 10 )$$

where τ is the mean transit time through the venous compartment. For τ=0 and SaO₂=100%, equation (10) reduces to equation (6). In adults τ has been estimated to be in a range between 3 to 4 seconds, although other values may be possible.

Using parameters as described above, additional parameters may be computed for each subject. In some aspects, a channel with the strongest CBF and SO₂ responses among the four common DCS and CWNIRS channels may be used. Specifically, a CBF/CMRO₂ coupling ratio n, defined as the ratio of the fractional change in CBF to the fractional change in CMRO₂ may be computed:

$$n = \frac{\% rCBF}{\% {rCMRO}_{2}} \qquad ( 11 )$$

In addition a flow/volume coefficient may also be computed as:

$$Φ = \frac{log ( rCBV )}{log ( rCBF )} \qquad ( 12 )$$

Eqn. (12) may be used to convert measured rCBF into rCBV using Grubb's law (which assumes a constant relationship between rCBF and rCBV), which may be compared with measured rCBV values.

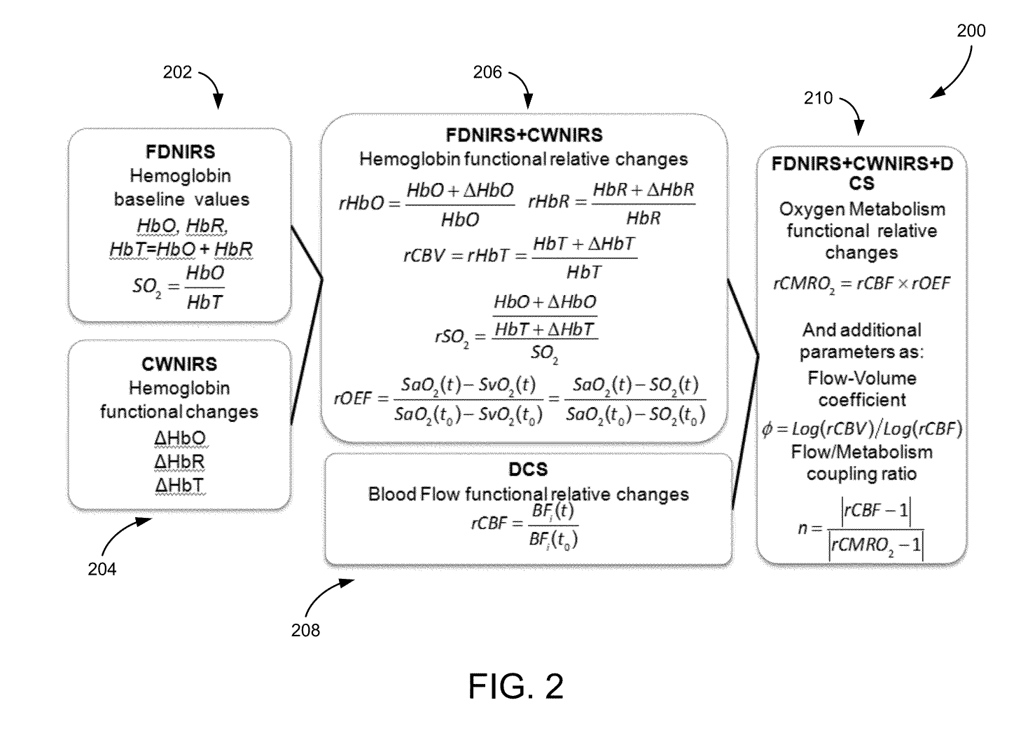

Referring to FIG. 2 a schematic diagram is shown illustrating a process 200 of combining physiological data acquired using the FDNIRS, CWNIRS, and DCS techniques, as described, to generate metabolic and hemodynamic parameters. In particular, hemoglobin base parameters obtained at process block 202 may be combined with hemoglobin functional changes parameters obtained at process block 204 to generate hemoglobin functional relative changes parameters at process block 206. The hemoglobin functional relative changes parameters from process block 206 may then be combined with blood flow functional relative changes parameters obtained at process block 208 to generate oxygen metabolism functional relative changes parameters at process block 210. Additionally, at process block 210, flow-volume and flow-metabolism coupling ratio coefficients may also be determined, as described.

Hemodynamic and metabolic parameters, as described above in Eqns. (1) through (10) are all functions of time, each parameter responds to a brain state of a subject, such as bursts during burst suppression in a manner similar to an external stimulus. Thus, given the burst times, it is possible to estimate a hemodynamic or metabolic response function, which quantifies the time course of the hemodynamic or metabolic parameter in response to a burst during burst suppression. Let the index i denote any of the time-varying hemodynamic or metabolic quantities described above, and let hᵢ(t) denote the corresponding hemodynamic/metabolic response function (“HRF”). Let yᵢ=(^{t}) represent the calculated hemodynamic/metabolic values described in equations (1) through (10) associated with the index i. Let u(t) denote the time series of burst suppression indicator values, equal to 1 during a burst period, and equal to 0 during a suppression period. These burst and suppression periods can be identified by any number of methods, including bandpass filtering and thresholding. All measurements and variables may be sampled at the same sampling rate. Given a total of T observations and M values for the HRF, one can re-write these functions in vector and matrix form as follows:

$${y}_{i} = [ \begin{matrix}{y}_{i} ( t ) \\ ⋮ \\ {y}_{i} ( t + T - 1 )\end{matrix} ] , \\ U = [ \begin{matrix}u ( t ) & … & u ( t + M - 1 ) \\ u ( t + 1 ) & … & u ( t + M ) \\ ⋮ & … & ⋮ \\ u ( t + T - 1 ) & … & u ( t + T + M - 2 )\end{matrix} ] , \\ {h}_{i} = [ \begin{matrix}{h}_{i} ( 1 ) \\ ⋮ \\ {h}_{i} ( M )\end{matrix} ] . \qquad ( 13 )$$

Then, the relationship between the measurements, indicators, and HRF can be modeled as follows,

yᵢ=Uhᵢ+ε (14)

where ε represents a Gaussian white noise term with zero mean and variance σ². The HRF and its variance can then be estimated using ordinary least squares technique, namely:

ĥᵢ=(U^{T}U)⁻¹U^{T}yᵢ

var(ĥᵢ=σ²(U^{T}U)⁻¹ (15)

In this manner, HRFs for desirable metabolic and hemodynamic parameters, as described, may be generated for use in determining a brain state of a subject.

In some instances, it may be desirable to characterize time-varying changes in the hemodynamic and metabolic response functions hᵢ. This could be accomplished in a number of ways. For example, an initial estimate of the hemodynamic response function can be performed using Eqns. (13), (14), and (15), defined as hᵢ(0). The hemodynamic or metabolic response function can then be modeled as a time-varying function, hᵢ(t), whose temporal evolution is governed by a linear state-space model, such as a random walk, according to:

hᵢ(t)=hᵢ(t−1)+w(t) (16)

where w(t) is an independent, identically-distributed Gaussian noise process with variance Σ_{w}=σ_{w}²I. An observation equation may then be written relating the measured data to the time-varying hemodynamic response:

yᵢ(t)=u^{T}(t)hᵢ(t)+v(t)

u^{T}(t)=[u(t) . . . u(t+M−1)] (17)

where v(t) is an independent, identically-distributed Gaussian noise process with variance Σᵥ=σᵥ²I. In this representation, the solution for the time-varying hᵢ(t) may then be obtained using any number of techniques appropriate for linear state-space systems, such as a Kalman filter or a fixed-lag smoother. The unknown parameters σ_{w}² and σᵥ² can be estimated from the data using any suitable methods

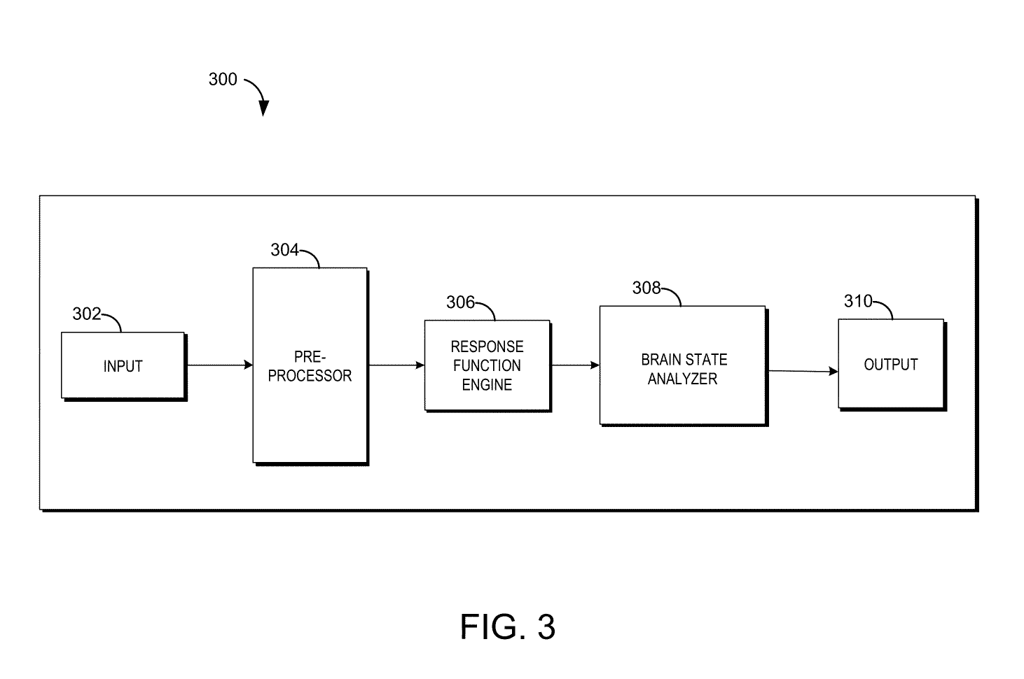

Specifically referring to FIG. 3, an example system 300 for carrying out steps for determining a brain state of a subject, as described above, is illustrated. The system 300 includes an input 304, configured to receive physiological data from a sensor array in communication with the system 300 via a wired or wireless connection. The received physiological data includes optical data, such as FDNIRS, CWNIRS and DCS data, as well as electrophysiological data, such as EEG data. The system 300 also includes a pre-processor 304, configured for pre-processing or conditioning the acquired physiological data. In particular, the pre-processor 304 is configured to carry out any number of pre-processing steps, such as assembling the received physiological data into time-series signals and performing a noise rejection step to filter any interfering signals associated with the acquired physiological data. The pre-processor is also configured to receive an indication via the input 302, such as information related to administration of an anesthesia compound or compounds, and/or an indication related to a particular subject profile, such as a subject's age, height, weight, gender, or the like, as well as drug administration information, such as timing, dose, rate, and the like.

The system 300 also includes a response function engine 306, configured to compute desired metabolic and hemodynamic parameters, and corresponding response functions, as described, which may be performed in parallel, in succession or in combination, using data received from the pre-processor 304. Computed response functions, among other information, may then be relayed to a brain state analyzer 308 designed to carry out steps necessary for determining a brain state, such as a metabolic or hemodynamic state, of a subject, as described. Information related to the determined state(s) may then be relayed to the output 310, along with any other desired information, in any shape or form. For example, the output 310 may include a display configured to provide information related to a current brain state, and/or future brain state based on the indication provided. In addition, the output 310 may include information regarding an efficacy of a treatment, or may include instruction for an adjustment of treatment.

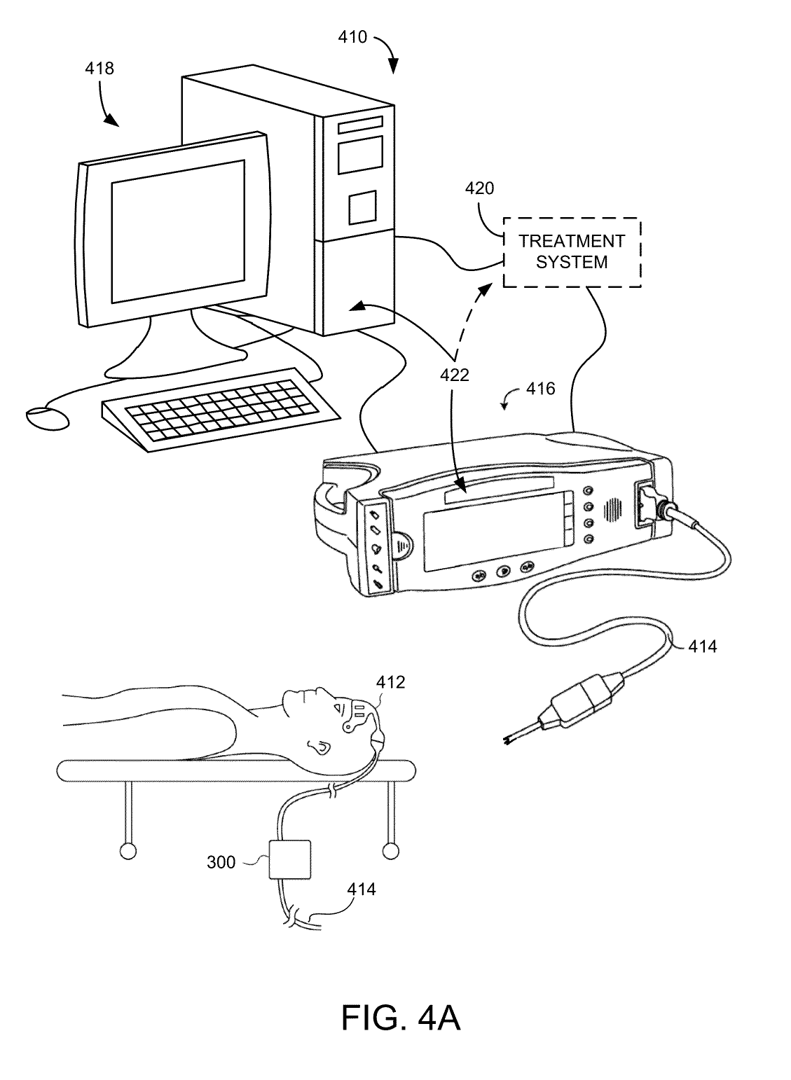

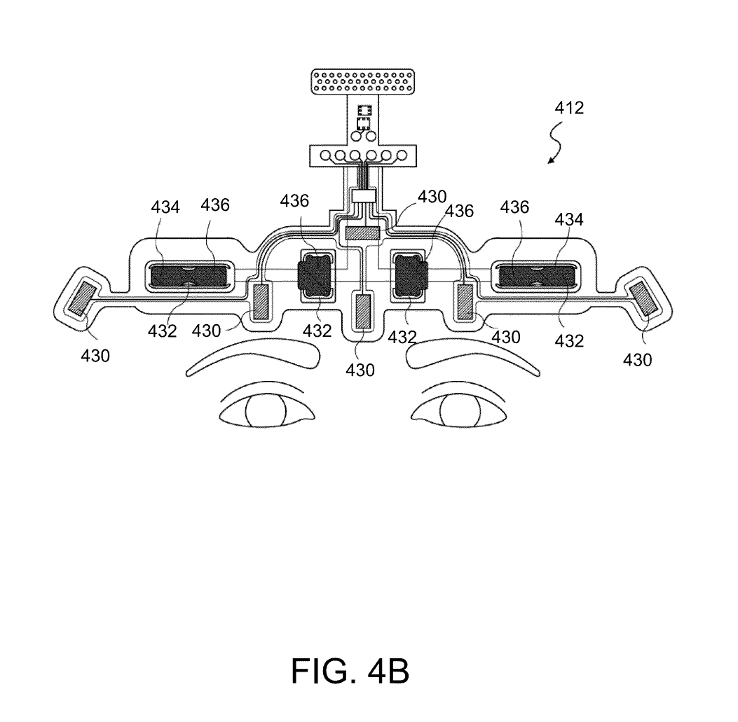

Specifically referring to FIG. 4A, an example system 410 in accordance with the present disclosure is illustrated, for use in monitoring and/or controlling a state of a subject during a medical procedure, or as result of an injury, pathology or other condition. In some aspects, the system 410 could be used to guide or control medically-induced coma, anesthesia, or sedation. In other aspects, the system 410 could be used to guide or control medically-induced hypothermia, for instance during hypothermia treatment after cardiac arrest, or during cardiac surgery.

The system 410 includes a subject monitoring device 412 that includes multiple sensors, including electrophysiological sensors, such as EEG sensors, and optical sensors, such as blood oxygenation sensors, and so forth. However, it is contemplated that the subject monitoring device 412 may incorporate other sensors including blood oxygenation sensors, ECG sensors, temperature sensors, acoustic respiration monitoring sensors, and so forth. As shown in FIG. 4B, one realization of this design incorporates a frontal array of electrophysiological sensors 430 and optical sensors 432. The optical sensors include a number of light sources 434 and light detectors.

The subject monitoring device 412 is connected via a cable 414 to communicate with a monitoring system 416, which may be a portable system or device, and provides input of physiological data acquired from a subject to the monitoring system 416. In some aspects, the subject monitoring device 412 may be in communication with a system 300 configured for determining and/or relaying information a brain state of a patient using hemodynamic and metabolic parameters obtained from optical data, as described. Also, the cable 414 and similar connections can be replaced by wireless connections between components. As illustrated, the monitoring system 416 may be further connected to a dedicated analysis system 418. Also, the monitoring system 418 and analysis system 418 and system 300 may be integrated.

The monitoring system 416 may be configured to receive raw signals acquired by the sensors and assemble, and even display, the raw signals as waveforms. Accordingly, the analysis system 418 may receive the waveforms from the monitoring system 416 and, as will be described, analyze the waveforms and signatures therein, determine a brain state of the subject, such as a burst suppression state, based on the analyzed waveforms and signatures, and generate a report, for example, as a printed report or, preferably, a real-time display of signature information and determined state. However, it is also contemplated that the functions of monitoring system 416, analysis system 418, and system 300 may be combined into a common system.

In some configurations, the system 410 may also include a treatment delivery system 420. The treatment delivery system 420 may be coupled to the analysis system 418 and monitoring system 416, such that the system 410 forms a closed-loop monitoring and control system. Such a closed-loop monitoring and control system in accordance with the present disclosure is capable of a wide range of operation, and may include a user interface 422, or user input, to allow a user to configure the closed-loop monitoring and control system, receive feedback from the closed-loop monitoring and control system, and, if needed reconfigure and/or override the closed-loop monitoring and control system.

In some configurations, the treatment delivery system 420 may include a drug delivery system not only able to control the administration of anesthetic compounds for the purpose of placing the subject in a state of reduced consciousness influenced by the anesthetic compounds, such as general anesthesia or sedation, but can also implement and reflect systems and methods for bringing a subject to and from a state of greater or lesser consciousness. In other configurations the treatment delivery system 420 may include a hypothermia treatment system. Other treatments may be administered or facilitated by the treatment delivery system 420 as well.

Certain applications could be facilitated by providing specific information output via any number of graphical displays. For example, systems, as provided by the present disclosure, may include configurations whereby constructed EEG waveforms and optical time-series could be displayed concurrently, such that the temporal relationship between raw or processed signals could be appreciated by monitoring physicians or nurses. The time-varying estimates of hemodynamic or metabolic response functions could be displayed alongside a prototype or reference waveform associated with desired hemodynamic or metabolic responses. Statistically-significant deviations from this reference waveform could signal an auditory or visual alert intended to prompt clinical action. In addition, such systems may continuously or periodically store the estimated hemodynamic and metabolic response functions, say at an interval of minutes, tens of minutes, or an hour. This stored record of hemodynamic and metabolic response functions could then be recalled and displayed to show the history of such responses throughout a patient's treatment, procedure, or stay within the intensive care unit. This historical display could be used to make prognostic assessments for a patient's course of recovery. Numerical parameters, such as the burst suppression rate or burst suppression probability, or the flow-metabolism coupling ratio, could also be displayed and updated periodically, say every few seconds, or with the occurrence of a burst.

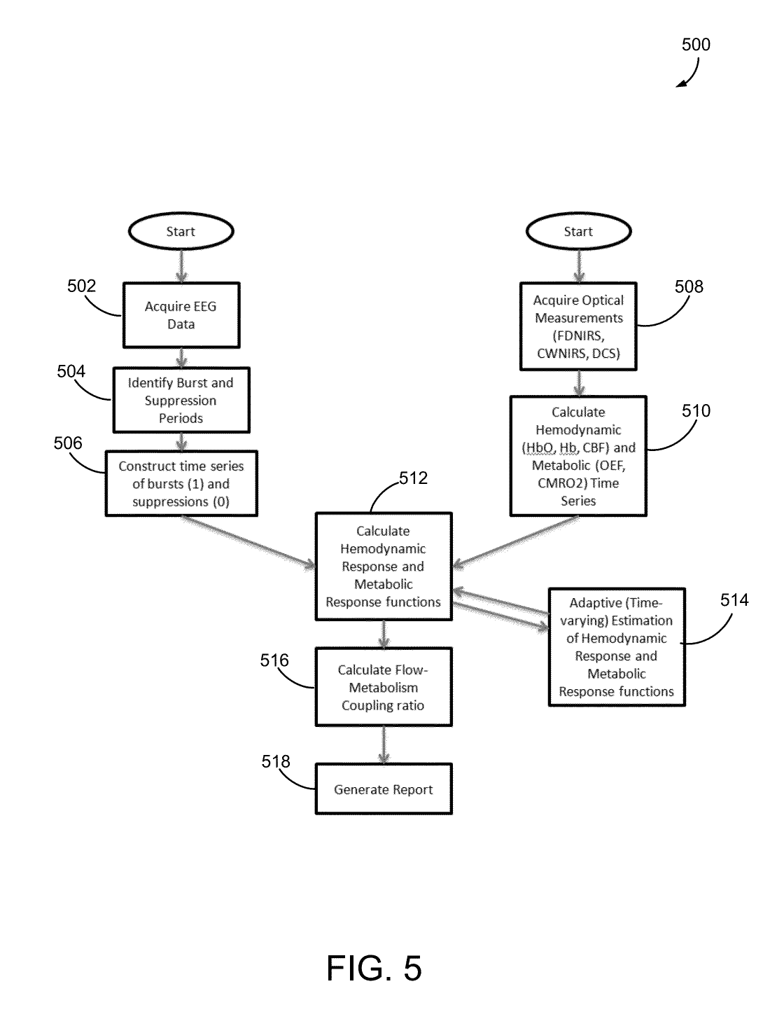

Turning to FIG. 5, a flowchart is shown setting forth steps for a process 500 for monitoring and controlling a brain state of a subject. The process may begin at process block 502 where EEG data, is acquired using a single sensor or a plurality of sensors, and pre-processed in any manner. At process block 504, the EEG data may then be processed to identify burst and suppression periods, using any autonomous or semi-autonomous techniques suitable. Assembling the EEG data as time-series signals, at process block 506, burst periods are assigned a value of “1,” and suppression periods are assigned a value of “0,” as described. At process block 508, optical data from optical sensors may be acquired, either substantially concurrent with or following process block 506. At process block 510, the optical data may then be used to calculate hemodynamic and metabolic time series, as described in Eqns. (1) through (10), and shown in FIG. 2. The outputs from process blocks 506 and 510 may then be combined at process block 512 to calculate hemodynamic response and metabolic response functions, as described in Eqns. (13) through (15). Then, at process block 514, the hemodynamic response and metabolic response functions can be estimated in a time-varying manner, as described. At process block 516, the hemodynamic and metabolic response functions can be used to calculate the flow-metabolism coupling ratio, as described in Eqn. (11). Then, at process block 518, a report, of any shape or form, including information related to the estimated response functions, may be generated. In some aspects, information related to parameters and respective response functions may be relayed to any clinician, or control system, for use to control an administration of a treatment. For example, using the response function an indication may be provided with respect to a target burst suppression state, including information regarding a likelihood or trajectory of a recovery, or an efficacy of a drug therapy or treatment.

In some applications, systems and methods, as provided by the present disclosure, may be used to monitor cerebral hemodynamic responses and metabolic responses in a number of different operating room procedures. For instance, during major cardiac surgery, patients can be placed into a state of burst suppression with a combination of cooling and general anesthesia, to reduce brain metabolism and provide brain protection. In such procedures, the hemodynamic and metabolic responses to bursts could be used to track changing brain cerebrovascular function and metabolism. For instance, with increased cooling and burst suppression, reduced amplitude responses in CMRO₂, CBF, and HbO and Hb to bursts could indicate reduced metabolism associated with brain protection. Increases in these parameters could indicate changing brain metabolism and health during surgery, and could prompt clinical intervention, such as increased cooling or efforts to increase brain perfusion. Furthermore, changes in the flow-metabolism coupling ratio could be used to monitor the balance of cerebral flow and metabolism. For instance, decreasing flow-metabolism coupling ratio would suggest that the brain is receiving inadequate flow relative to metabolism. This could prompt clinical intervention, such as increased cooling, or efforts to increase brain perfusion. During carotid end-arterectomy surgery, for instance, left-right asymmetries in hemodynamic response parameters or metabolism could indicate reduced perfusion, and could prompt clinical intervention, such as installation of a shunt.

In other applications, systems and methods, as provided by the present disclosure, may be used to provide patient monitoring in intensive care situations and settings, where patients can be in a burst suppression brain state for a variety of reasons. For example, post-anoxic coma patients often remain in burst suppression during coma. Also, patients with epilepsy or traumatic brain injuries can be placed in medically-induced coma using general anesthetic drugs such as propofol. Changes in burst-induced hemodynamic or metabolic responses could indicate improving or declining brain health, and could prompt clinical intervention, or guide prognosis. For instance, a coma patient with steadily improving hemodynamic responses, CMRO₂, and flow-metabolism coupling ratio might have a greater likelihood of survival, which then might dictate continued medical treatment to accelerate or facilitate recovery. On the other hand, a patient whose hemodynamic responses, CMRO₂, and flow-metabolism coupling ratio decrease, could indicate worsening of condition that would require intervention, or would suggest a negative prognosis that could prompt cessation of care. For medically-induced coma, these hemodynamic and metabolic responses could be used to find some optimal state of reduced brain metabolism, where for instance cerebral blood flow could be maximized relative to metabolism, resulting in a state where the flow-metabolism coupling ratio was high. Similarly, in patients with epilepsy, the size of hemodynamic responses could be used to infer the level of seizure activity present within bursts, and could be used to determine a point at which to end the medically-induced coma, say when metabolic or hemodynamic responses return to normal levels.

### Example

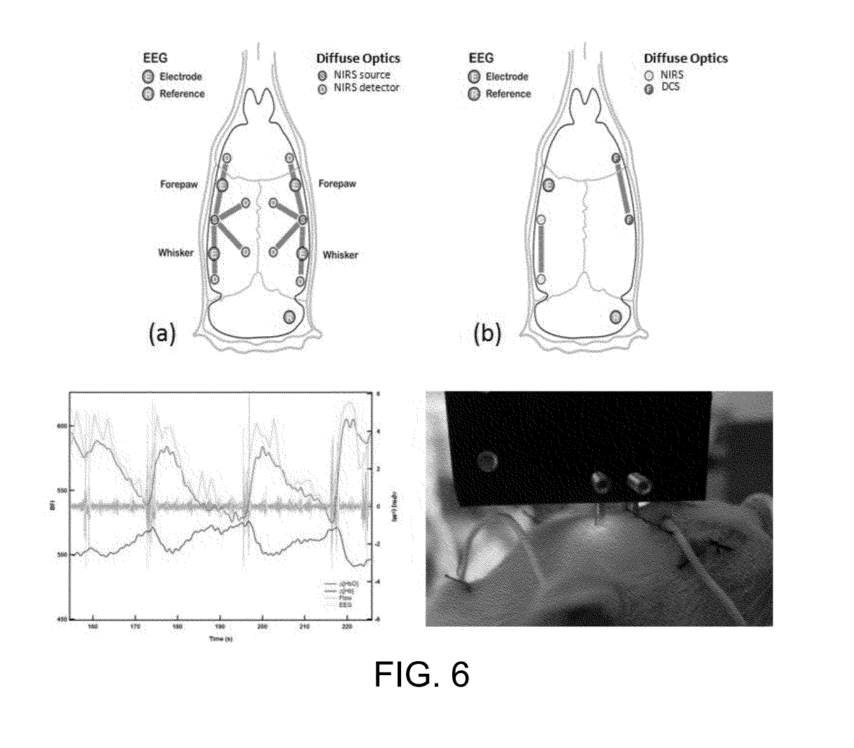

Experiments were carried out on rats, using both invasive and non-invasive measurements. All rats were tracheotomized and mechanically ventilated with 100% oxygen plus isoflurane. Body temperature was maintained at 37-degrees Celsius through external heating. Oxy- and Deoxy-hemoglobin changes were measured using continuous wave near infrared spectroscopy (CWNIRS) acquired at a rate of 50 Hz, while cerebral blood flow was measured using diffusion correlation spectroscopy (DCS), acquired at a rate of 1 or 4.5 Hz. EEG was recorded at a sampling rate of 1 kHz. The configuration of sensors is shown in FIG. 6. Blood pressure, body temperature, ventilation pressure, and end-tidal CO₂ were continuously recorded. Isoflurane concentrations were varied between 1% and 3.5% to induce different rates of burst suppression.

As shown in FIG. 2, NIRS and DCS was used in combination to obtain oxy-hemoglobin (HbO), deoxyhemoglobin (HbR), cerebral blood flow (CBF), oxygen extraction ratio (OEF), and cerebral metabolic rate of oxygen (CMRO₂). NIRS data was employed from both frequency domain (FDNIRS) and continuous wave (CWNIRS) measurements to obtain information on absolute and relative hemoglobin values, respectively.

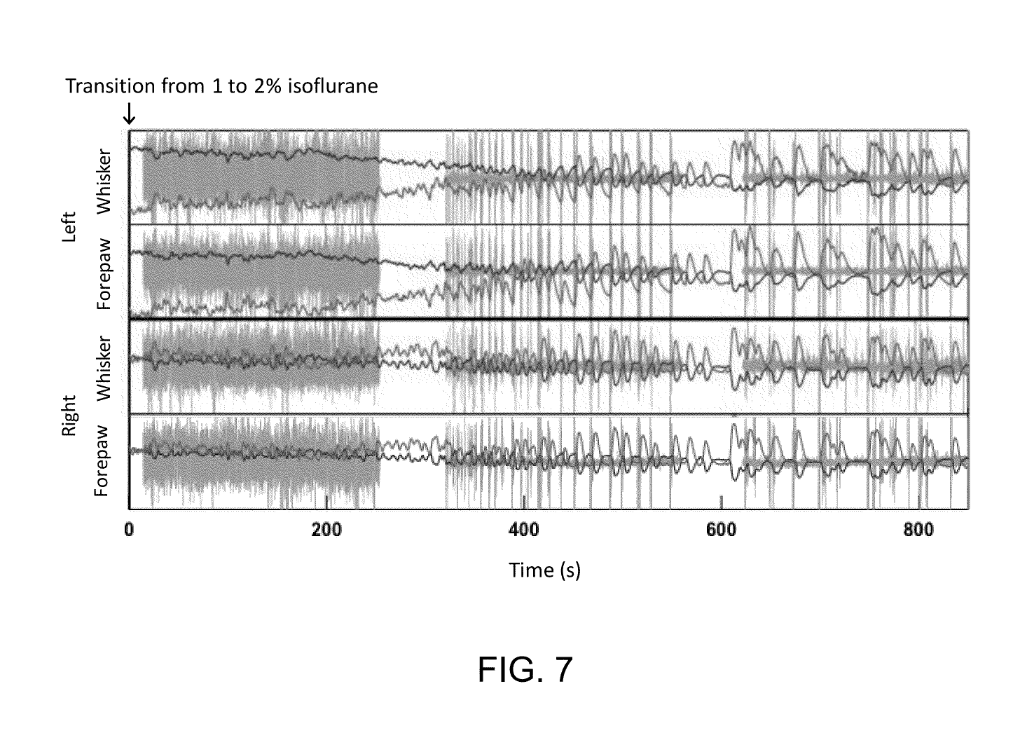

FIG. 7 shows HbO (red) and Hb (blue) time series in relation to EEG (gray) during the transition from 1 to 2% isoflurane. As the isoflurane concentration increases, the suppression periods become longer, and the bursts become less frequent. The close relationship between bursts and HbO and Hb time series is readily observed, where each burst is associated with a sharp increase in HbO, and a similar decline in Hb, consistent with the typical changes observed during functional activation.

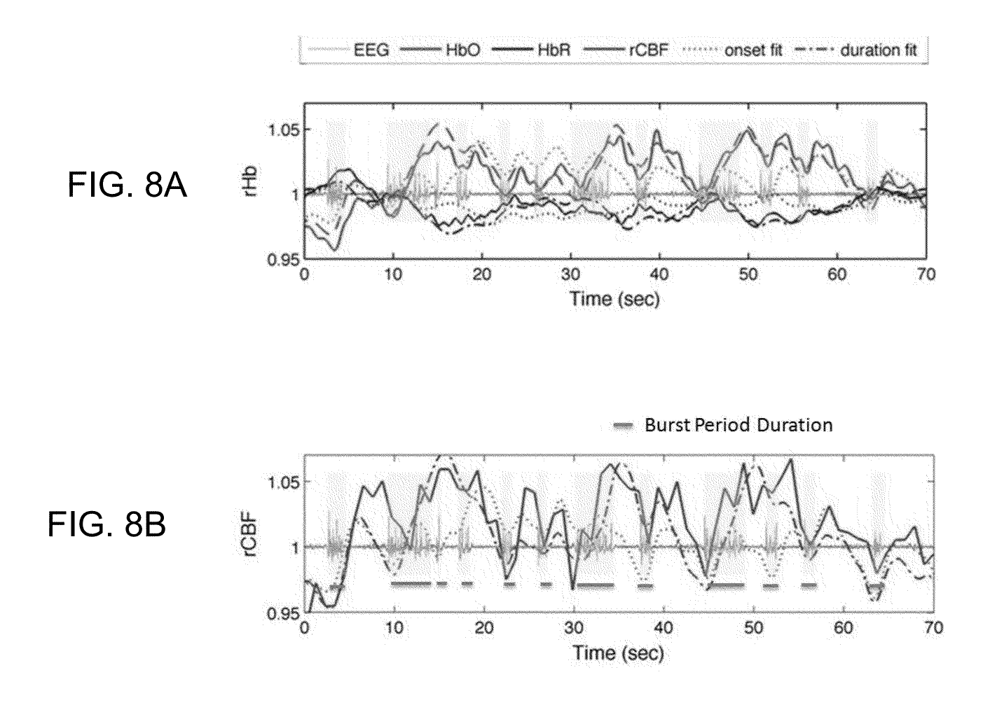

FIG. 8 shows a more detailed view of HbO and HbR in relation to burst suppression (FIG. 8A), as well as rCBF (FIG. 8B). This figure compares the HRF fit or prediction using the full burst indicator function specified above (“duration fit”), versus one where only the onset of the burst is accounted for (“onset fit”). The indicator function u(t) is constructed from the EEG time series by identifying burst and suppression periods, as denoted in FIG. 8B, and assigning a “1” to burst periods, and a “0” to suppression periods. Using the full duration of the burst to construct the indicator function u(t), rather than just the onset of the burst, produces a more accurate representation of the observed HbO, Hb, and rCBF time series.

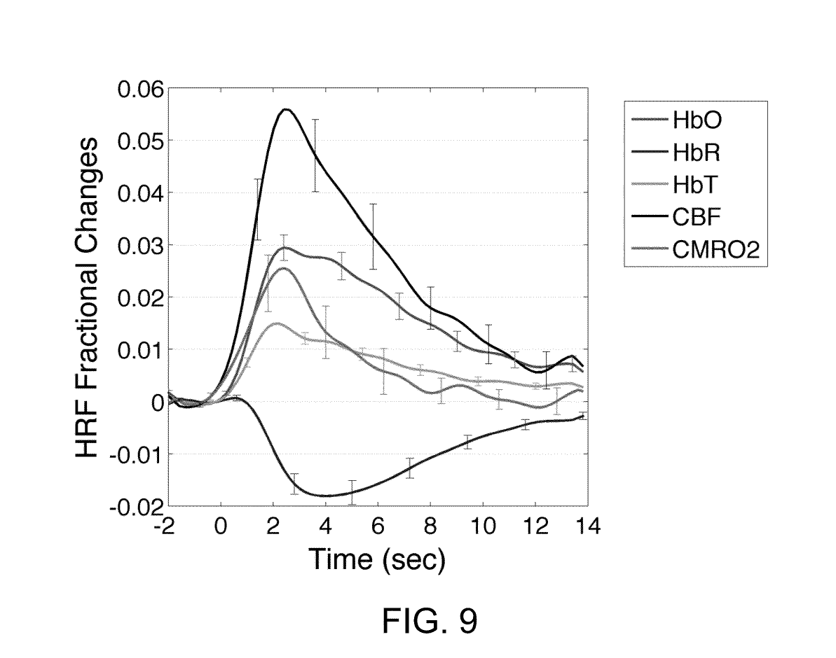

FIG. 9 shows the estimated burst-suppression HRF's for HbO, HbR, HbT, CBF, and CMRO₂. The fractional changes for these quantities vary between −2% to +6%, reflecting a large change in response to each burst. The response persists over a 14 second period, consistent with the typical duration for stimulus-evoked responses.

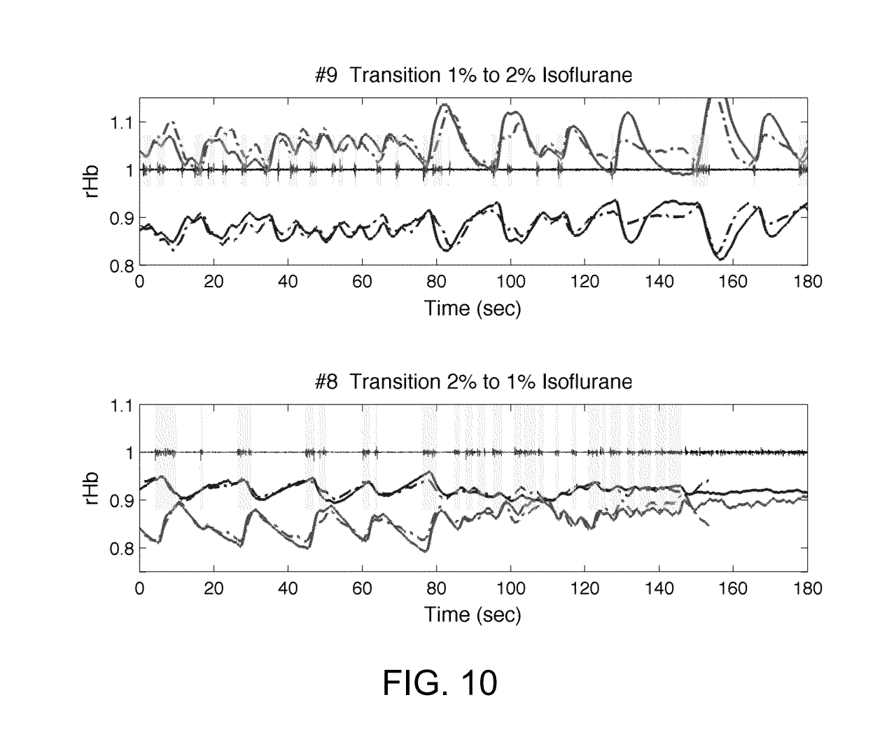

FIG. 10 provides another detailed view of HbO (red), Hb (blue), and their HRF predictions (dotted lines) during increasing (top) and decreasing (bottom) concentrations of isoflurane.

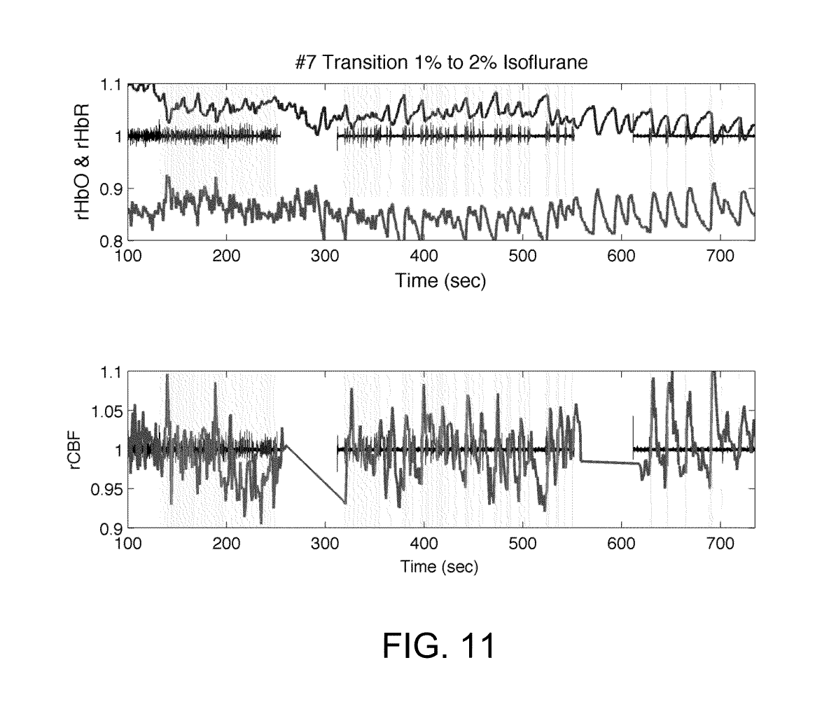

FIG. 11 shows HbO (red), Hb (blue), and CMRO₂ (green) during increasing concentrations of isoflurane.

Embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

1. A system for monitoring and controlling a state of a subject, the system comprising:
an input configured to receive physiological data from a plurality of sensors coupled to the subject, the plurality of sensors including electrophysiological sensors and optical sensors;
at least one optical source configured to direct light in a range of wavelengths to at least one portion of a subject's anatomy;
at least one processor configured to:
acquire the physiological data from the plurality of sensors positioned on a subject;
assemble, using the physiological data from the electrophysiological sensors, a time-series signal indicative of a brain activity of the subject;
identify, using the time-series signal, a burst suppression state described by a burst suppression period and a burst period;
compute, using the physiological data from the optical sensors, parameters associated with the burst suppression state, the parameters indicative of least one of a metabolic process and a hemodynamic process;
estimate, using the parameters, time-series signal, and burst period, a response function describing a time course of the parameters correlated with a burst during the burst suppression period; and
generate a report indicative of the response function.

2. The system of claim 1, wherein the range of wavelengths includes a near-infrared range between 650 and 950 nanometers.

3. The system of claim 1, wherein the at least one optical source is configured to probe at least one of a static property and a dynamic property of biological tissue within at least one portion of the a subject's anatomy, wherein the static property includes a tissue absorption and a tissue scattering, and the dynamic property includes a motion of scatterers.

4. The system of claim 1, wherein the system is further configured to acquire the physiological data using at least one of a frequency domain near infra-red spectroscopy (“FD-N IRS”) technique, a continuous-wave near-infrared spectroscopy (“CW-NIRS”) technique, and diffusion correlation spectroscopy (“DCS”) technique.

5. The system of claim 1, wherein the at least one processor is further configured to compute at least one of an oxy-hemoglobin (“HbO”) parameter, a deoxyhemoglobin (“HbR”) parameter, a cerebral blood flow (“CBF”) parameter, an oxygen extraction (SO₂) parameter, an oxygen fraction (“OEF”) parameter, a cerebral flow volume (“CFV”) parameter, a cerebral metabolic rate of oxygen (“CMRO₂”) parameter, a flow-volume parameter, and a flow-metabolism coupling ratio parameter.

6. The system of claim 1, wherein the at least one processor is further configured to correlate the response function with a brain state of the subject and wherein the report indicates the brain state of the subject.

7. The system of claim 6, wherein the brain state of the subject is defined by at least one of a metabolic characteristic and a hemodynamic characteristic.

8. The system of claim 6, wherein the at least one processor is further configured to generate a target burst suppression state using the state, the response function and an indication received from the input, the indication including at least one of a patient characteristic, an anesthetic dose, an anesthetic administration time, an anesthetic infusion rate, a temperature, and a temperature rate.

9. The system of claim 1, wherein the at least one processor is further configured to control an administration of a treatment to achieve the generated target burst suppression state.

10. The system of claim 9, wherein the treatment includes one of a hypothermia treatment and an anesthesia treatment.

11. A method for monitoring a brain state of a subject, the method comprising:
acquiring physiological data from a plurality of sensors positioned on the subject, the plurality of sensors including electrophysiological sensors and optical sensors;
assembling, using the physiological data from the electrophysiological sensors, a time-series signal indicative of a brain activity of the subject;
identifying, using the time-series signal, a burst suppression state described by a burst suppression period and a burst period;
computing, using the physiological data from the optical sensors, parameters associated with the burst suppression state, the parameters indicative of least one of a metabolic process and a hemodynamic process;
estimating, using the parameters, time-series signal, and burst period, a response function describing a time course of the parameters correlated with a burst during the burst suppression period; and
generating a report indicative of the response function.

12. The method of claim 11, wherein the range of wavelengths includes a near-infrared range between 650 and 950 nanometers.

13. The method of claim 11, wherein method further comprises acquiring physiological data using at least one of a frequency domain near infra-red spectroscopy (“FD-N IRS”) technique, a continuous-wave near-infrared spectroscopy (“CW-NIRS”) technique, and diffusion correlation spectroscopy (“DCS”) technique.

14. The method of claim 11, wherein the method further includes computing at least one of an oxy-hemoglobin (“HbO”) parameter, a deoxyhemoglobin (“HbR”) parameter, a cerebral blood flow (“CBF”) parameter, an oxygen extraction (SO₂) parameter, an oxygen fraction (“OEF”) parameter, a cerebral flow volume (“CFV”) parameter, a cerebral metabolic rate of oxygen (“CMRO₂”) parameter, a flow-volume parameter, and a flow-metabolism coupling ratio parameter.

15. The method of claim 11, wherein the method further comprises correlating the response function with a brain state of the subject and wherein the report indicates the brain state of the subject.

16. The method of claim 15, wherein the brain state of the subject is defined by at least one of a metabolic characteristic and a hemodynamic characteristic.

17. The method of claim 15, wherein method further comprises generating a target burst suppression state using the state, the response function and an indication received from the input, the indication including at least one of a patient characteristic, an anesthetic dose, an anesthetic administration time, an anesthetic infusion rate, a temperature, and a temperature rate.

18. The method of claim 11, wherein the method further comprises controlling an administration of a treatment to achieve the target burst suppression state.

19. The method of claim 18, wherein the treatment includes one of a hypothermia treatment and an anesthesia treatment.

20. A method for monitoring and controlling a brain state of a subject, the method comprising:
acquiring physiological data from a plurality of sensors positioned on the subject, the plurality of sensors including electrophysiological sensors and optical sensors;
assembling, using the physiological data from the electrophysiological sensors, a time-series signal indicative of a brain activity of the subject;
identifying, using the time-series signal, a burst suppression state described by a burst suppression period and a burst period;
computing, using the physiological data from the optical sensors, parameters associated with the burst suppression state, the parameters indicative of least one of a metabolic process and a hemodynamic process;
estimating, using the parameters, time-series signal, and burst period, a response function describing a time course of the parameters correlated with a burst during the burst suppression period; and
controlling an administration of a treatment using the response function to achieve a target burst suppression state.

21. The method of claim 20, wherein the range of wavelengths includes a near-infrared range between 650 and 950 nanometers.

22. The method of claim 20, wherein method further comprises acquiring physiological data using at least one of a frequency domain near infra-red spectroscopy (“FD-N IRS”) technique, a continuous-wave near-infrared spectroscopy (“CW-NIRS”) technique, and diffusion correlation spectroscopy (“DCS”) technique.

23. The method of claim 20, wherein the method further includes computing at least one of an oxy-hemoglobin (“HbO”) parameter, a deoxyhemoglobin (“HbR”) parameter, a cerebral blood flow (“CBF”) parameter, an oxygen extraction (SO₂) parameter, an oxygen fraction (“OEF”) parameter, a cerebral flow volume (“CFV”) parameter, a cerebral metabolic rate of oxygen (“CMRO₂”) parameter, a flow-volume parameter, and a flow-metabolism coupling ratio parameter.

24. The method of claim 20, wherein the method further comprises correlating the response function with a brain state of the subject.

25. The method of claim 24, wherein the method further comprises generating a report indicative of the brain state of the subject

26. The method of claim 24, wherein the brain state of the subject is defined by at least one of a metabolic characteristic and a hemodynamic characteristic.

27. The method of claim 15, wherein controlling the administration of the treatment includes receiving an indication from an input that includes at least one of a patient characteristic, an anesthetic dose, an anesthetic administration time, an anesthetic infusion rate, a temperature, and a temperature rate.

28. The method of claim 20, wherein the treatment includes one of a hypothermia treatment and an anesthesia treatment.
